(19) United States
(12) Patent Application Publication
(10) Pub. No.: US 2003/0054348 A1
(43) Pub. Date: Mar. 20, 2003

(54) Methods of identifying the activity of gene products

(76) Inventors: Arthur J. Blume, Annandale, NJ (US); Neil Goldstein, Maplewood, NJ (US); Renuka Pillutla, Bridgewater, NJ (US); Ku-Chuan Hsiao, Easton, PA (US); John Prendergast, Lawrenceville, NJ (US)

Correspondence Address: MORGAN & FINNEGAN, L.L.P., 345 Park Avenue, New York, NY, 10154-0053, US

(21) Appl. No.: 09/852,455

(22) Filed: May 9, 2001

Related U.S. Application Data
(60) Provisional application No. 60/202,912, filed on May 9, 2000.

(51) Int. Cl. C12Q 1/68; G01N 33/53; G01N 33/567

(52) U.S. Cl. 435/6; 435/7.1; 435/7.21

(57) ABSTRACT

Methods are disclosed for determining the activity and function of gene products based on their change to the phenotype of cells when contacted with a surrogate molecule identified through screening of a recombinant peptide library. By identifying peptides which bind gene products for which the function is not yet known, tools are provided for determining their function and obtaining drug candidates.

This application claims priority to U.S. provisional patent application Ser. No. 60/202,912 filed May 9, 2000 which is incorporated herein by reference in its entirety.

### FIELD OF THE INVENTION

This invention relates to a general method for identifying the activity or function of gene products by identifying peptide binding partners which cause a cellular response in cells expressing the gene products. Accordingly, this invention is useful for determining the influence which specific genotypes have on phenotypes. In particular, the invention is concerned with a method of obtaining peptides which bind to a target such as a novel gene product. Such peptides provide 1) sequences that may be used to identify the natural protein partner of the target, and 2) enable synthesis of peptides which alter the phenotype of cells expressing the target. This invention also relates to providing material useful for conducting competitive binding assays capable of identifying small molecules reactive with and modulatory of the target protein.

### BACKGROUND OF THE INVENTION

Present estimates of the number of different genes range over 30,000 and may read over 150,000 if one considers splicing varients. Despite rapid progress at identifying genes based on analysis of the human genome, progress identifying the activity and function of the gene products lags significantly behind. A number of methods have been reported for connecting specific genes with specific diseases or conditions of pharmaceutical interest. One general method, referred to here as a genomic ‘knock-out’, eliminates, physically or by mutation, single base deletion or insertion, the gene in question. The earliest of these knock-outs in animals have been done in such a fashion that function can be lost in all cells derived from a single fertilized egg. Genomic knock-outs, done in cells as well as animals have revealed the functions of many genes.

Genomic knock-outs have severe limitations regarding providing useful pharmacological targets. These limitations result from the occurrence of the knock-out in many places at one time and an all or none event occurring very early in development whereas many diseases result from timed and or graded alteration in gene activity. Furthermore, the gene causing the phenotypic change often is not the best target for drug therapy, and little information may be gained by this procedure on the best drug target. Lastly, knowing the gene target does not necessarily provide the investigator with a simple tool for obtaining small organic molecules which act on the target of interest and are useful for animal phenotyping and as drug leads.

A second knock-out approach to elucidating gene function is the use of anti-sense nucleic acids to prevent translation of mRNA into functional proteins. In this approach, antisense molecules can be applied from without or within the cell. Although this method has the clear advantage of being controllable with respect to timing, and graded response, it suffers from the fact that mRNA and protein are not uniformly linked, therefore allowing a large degree of variation in expected protein level manipulation. In addition, antisense approaches are prong to non-specific artifacts which will confound the phenotypic effect.

Sequence analysis (i.e., mutation identification) or quantitation of mRNA expression, are other genomic approaches to determining gene function and phenotype relation. Both methods however are associated with severe limitation for discovering the phenotypic relationship. As noted above, RNA quantitation is too removed from protein activity for one to rely on such information. In addition, although mutations can provide an association between a gene and a specific phenotype or condition, most mutations result in all or nothing events much unlike many disease conditions of interest.

Recent information has made it clear that there are large networks of genes coding for products which appear to be interrelated. Knocking out one of such genes influences the level of expression of the other. These gene networks are being elucidated via DNA chip technology which allows for the simultaneous quantitation of mRNA from a very large number of genes. See U.S. Pat. No. 5,800,992 and WO 95/35505 which are incorporated herein by reference. Although this information and the data bases derived from them are powerful road maps of networking, they suffer in their inability to distinguish initial interactions from secondary interactions from nth interactions. Although repeated quantitative analysis over many time points may provide some of this information of primary, secondary and later levels of protein interactions, such increases in experimental number are costly and time consuming. Network information on proteins of known function is useful, but is much less useful on genes of unknown function.

Many gene products produce their effect by binding to one or more other peptides or proteins. Presently, there are few approaches for identifying a protein's partner, i.e., the protein with which the target gene product directly interacts. This information is critical as most direct protein:ligand interactions, whether non-covalent or covalent, have major consequences for protein activity, including signaling, information transfer within and between parallel signaling cascades, and molecular processing. Examples of such protein:ligand interactions may include those between an enzyme and its substrate, a ligand (peptide or not) and its receptor, or a transport protein and ligand. Regulatory proteins also often function through binding to a molecular partner. Partner information therefore includes knowing, for example, the ligand for its receptor, the substrate for a ‘kinase’ or a protease, the regulatory protein controlling mRNA translation or DNA transcription.

The classical approach to partner identification is to obtain the target and its partner in some sort of isolated complex. Newer approaches place target and partner in two fusion proteins such that when they are complexed a signal is generated and the fusion protein or its gene sequence is used to identify the partner. For example, the yeast two-hybrid system has been used for partner identification. While the yeast two-hybrid approach is popular, it has a number of inherent problems including a high potential for false positives, the inability to use non-protein targets such as mRNA or membrane bound/extracellular proteins and the inability to address postranslational modifications on a target. Moreover, systems based on fusion proteins in general, while powerful, are not easily applied to a very large number of genes of unknown function, as this becomes a random association problem and would necessitate a combinatorial approach covering all genes.

For the subclass of proteins which interact with nucleic acids, most are currently identified based on information on the nucleic acid:protein complex which exists either in soluble form, or in gels or via some type of genetic recombinant expression system. Use of such an approach for the very large number of nucleic acid interacting proteins requires extensive efforts.

Partner information is critical to developing a target binding assay capable of identify drug leads. Among the large number of assays that exist, there are in vitro and cellular ones, and many types of binding assay formats in each case. The vast majority of In vitro ones, contain a target and a ligand but a few require only target. Those without ligand, suffer from not being directed to any particular surface on the target and therefore will generate a high frequency of false positives, i.e. compounds which bind but do not cause a change in target activity. In the case of unknown gene targets, only the non-directed assay could be used which would mean a much larger effort at screening than desired.

Panning of unknown gene products with phage displayed libraries to find natural partners would not seem worthwhile as the published results of panning of known receptor genes with known partners have not shown surrogate peptides to have natural partner amino acid motifs, sequences etc. Panning of the EPO (erythropoietin) and TPO (thrombopoietin) receptors identified potent peptides, which after dimerization are active. However, these identified peptides have no natural TPO or EPO motifs and therefore fail to identify these proteins in database searches based on amino acid sequences.

Housey, U.S. Pat. No. 5,877,007, relates to methods and compositions for screening for compounds which inhibit or activate a protein of interest expressed by a cell relative to a control cell.

Picksley et al., U.S. Pat. No. 5,770,377, relates to methods of identifying compounds which interfere with the binding of an oncogene protein, such as MDM2, to p53.

Blume, U.S. Pat. No. 6,010,861 relates to methods and compositions for identifying drug candidates based on the ability of the drug candidate to compete with a reporter molecule identified from a recombinant library.

All of the publications discussed above are incorporated by reference herein in their entirety.

### SUMMARY OF INVENTION

The method presented in detail in this application greatly simplifies identifying a target protein's partners and the establishment of site directed assays for test compounds which can regulate the activity of the target protein and enable phenotyping in in vitro, cellular and animal model systems and provide drug leads as well. A target protein's partner includes all naturally occurring binding partners and any precusor polypeptides which may be modified post translationally. A target is any naturally occurring target which may be a peptide, a protein, a nucleic acid, a polysaccharide or a combination thereof. A target may be, for example, a receptor, a transport protein, a regulatory site.

In one embodiment, the method of the invention involves the isolation of peptides, preferably from a recombinant phage display peptide library, which bind to the protein and nucleic acid (NA) products of genes of unknown function and contain sufficient information to allow identification of the natural partner protein of the target and high affinity binding peptides. This method can be automated to increase the number of known and unknown genes and gene products that can be used as targets. The term gene products encompass any post translational modifications.

In one embodiment of the invention, a method of identifying the function of gene products is provided by detecting the phenotypic change in a cell or animal following contact of the gene product with a binding peptide.

In another embodiment of the invention, the function of a binding peptide and its corresponding gene product is obtained through analysis of sequence data bases of naturally occurring protein or nucleic acid sequences. Homology of binding peptides identified from a library which bind a novel gene product, with a known peptide of known function, provides relevant information for determining the function of the novel gene product.

In another embodiment, the invention relates to a method for determining the activity of a gene product comprising the steps of 1) expressing the gene product in at least one cell type in which the gene product is active; 2) contacting the cells with a ligand known to bind the gene product; and 3) detecting a change in phenotype in the cells in which the gene product is active.

Thus, this invention provides means for identifying peptide ligands capable of activating or inhibiting gene products through their ability to bind to such gene products as well as the activity and function of the gene products themselves. The identification of active peptide ligands also provides means for identifying other molecules, preferably small organic molecules, which also are active at the sites at which the peptide ligands bind and which therefore are useful as drug candidates. This invention provides methods for identifying the activity of both binding partners, i.e., ligand and receptor, of gene products which together result in a phenotypic change.

Peptide binding ligands identified through this invention directly enable phenotyping studies in various systems, including cell, tissue, and simple organism, of surface and intracellular targets. Attachment of cell-penetrating peptide sequences to the peptide binding ligands provides a means for detecting intracellular action of the peptide binding ligand. For example, the reagent BioPORTER® from Gene Therapy Systems may be used to deliver peptide.

The present invention also provides a method to simplify and quicken the establishment of high through-put screening system (HTS) formats of competition binding assays that can identify small organic molecules and other test substances which are reactive with the surfaces on unknown targets and one capable of modifying their activity. This can be used to facilitate phenotyping in more complex models such as organisms and animals and eventually provide leads for drug development.

### DETAILED DESCRIPTION OF THE INVENTION

In one embodiment of the invention, the method involves panning of unknown gene protein products or other targets such as regulatory mRNA domains with phage displayed libraries of random peptides and obtaining a set of peptides which bind to such targets. Libraries included fully randomized libraries as well as libraries which contain fixed amino acids at particular positions among the other randomized amino acids. The number of peptide binders obtained may range from about 10 sequences to the order of 100s of sequences. More complex identification motifs may require obtaining a larger number of sequences. The peptide binders are sequenced and used individually or as consensus motifs to search for genes with expressed proteins of matching amino acid sequence. Soluble binding peptide ligands with and without penetrating peptide additions, are obtained from those which contain natural gene motifs or recurring novel sequences via synthetic or recombinant methods. To assess their activity and to identify their function either as gene products themselves, or of gene products to which they bind, they are applied to cells identified to express the target gene. Phenotypic changes including morphological, biochemical, genetic or immunological changes, other than changes in the target protein itself are then observed. The peptide binding ligands may then be labeled and used in competitive site directed assays for small molecules which interact at a regulatory domain of the target protein and as described in U.S. Pat. No. 6,010,861, incorporated herein by reference.

Another embodiment of the invention is a method of identifying a naturally occurring binding partner or precursor for a target by identifying an amino acid sequence motif which confers detectable binding properties of a peptide by screening a library of expressed amino acid sequences for binding of members to the target, identifying amino acid sequence motifs and comparing the identified amino acid sequence motifs to known amino acid sequences of a genome to identify a naturally occurring binding partner or precursor for said target. Motifs are patterns of amino acids common to the amino acids of the surrogates and the naturally occurring partner which may contain contact sites for the target. In addition, the nucleic acid sequence for identified naturally occurring binding partner or precursor may be determined

A further embodiment of the invention is a method of identifying an amino acid sequence motif which confers binding properties to a natural target by screening a library of expressed amino acid sequences for binding to the target, determining the amino acid sequence of the members of the library which bind to the target, and identifying as motifs common amino acid sequences.

Another embodiment of the invention is a method for determining the activity of a gene product by expressing said gene product in a cell, contacting the cells with a ligand which binds said gene product, and detecting a change in phenotype of the cells. In addition, the invention embodies a method of determining the phenotypic outcome of the expression of a gene product by expressing the gene product in cells, contacting said cells with an amino acid sequence that has a binding motif identified by screening members of a peptide library which bind to the target, and detecting a change in phenotype of the cells.

An additional embodiment of the invention is a method of identifying a naturally occurring binding partner or precursor for a target by identifying an amino acid sequences that bind the target by screening a library of expressed amino acid sequences and comparing the identified amino acid sequence to known amino acid sequences of a genome and identifying a gene product that possesses an amino acid sequence substantially similar to the identified amino acid sequence. The nucleic acid of the identified naturally occurring binding partner may also be determined.

The method of the invention has been tested using different types of targets wherein the partners and function are known. For example, one target was an extracellular protein growth and differentiation factor. Another target was a 5′-untranslated RNA domain. These tests are valid as neither target type has been panned before with peptide libraries to yield binding peptide-ligands or surrogates which have amino acid sequences sufficient to identify the target's natural and known partner. In the former case that partner is the factor's transmembrane receptor and in the later case a ribosomal binding protein, EIF2.

A match of a surrogate amino acid sequence, or at least a part thereof, with a natural sequence enables partner identification. Surrogates containing natural sequences likely interact with regulatory surfaces. Accordingly, these surrogates should be useful as antagonists and some may also be agonists. In either case, agonism and antagonist are readily assayable in a phenotyping study. Antagonism is directly assayable in the presence of the natural partner or after addition of the natural partner to target containing systems. For those surrogates which do not contain natural sequence motifs, one does not know, a priori, whether these entities will be regulatory as the nature of their target's binding surface is unknown. However, analysis of surrogate libraries indicates a very high percentage of binders found by panning methodologies are to regulatory surfaces. The peptide binders are identified by competition with natural ligands, partners or neutralizing antibodies. In order to take into account the possibility of nonregulatory surrogates, phenotyping would be done with a small number, about six (6), surrogates with unrelated sequence motifs and those which modified test systems phenotypes would be used initially for site directed assay development. It is possible that some surrogates would only function as antagonists of agonistic surrogates.

Present databases and computers allow rapid searches for partners based on surrogate sequences. Examples of available computer based programs to analyse sequences include BLAST, Patternfind, ExPASy, MEME

(Multiple EM for Motif Elicitation),

(http://meme.sdsc.edu/meme/website/intro.html, MAST (Motif Alignment and Search Tool, http://meme.sdsc.edu/mem/website/mast-intro.html).

(www.expasy.ch/) and ISREC (www.isrec.isb-sib.ch/software/software.html).

Identification of surrogates provides tools for partner identification, phenotyping and small molecule discovery. Given that a site directed assay is available at this early stage for the unknown target, high throughout screening allows the rapid identification of reactive small molecules of low target affinity. Combinatorial chemistry, allows for improvements in potency which would then provide small molecules for phenotyping and testing in animal models.

The method of determining the activity and functions of an unknown gene product is determined according to a preferred embodiment of the invention as follows:

1. An unknown full length gene is expressed and the gene product protein is isolated.

2. The gene product is then panned with a ≧20 mer surrogate library as described, for example in U.S. Pat. No. 6,010,861, and members of the library which bind the gene product are isolated and sequenced.

3. The sequences of a representative number of peptide binders are analyzed using a database such as BLASTp and then tBLASTn. These searches on protein and EST databases are directed at uncovering matches to known or unknown proteins and genes.

4. Overlapping ESTs are knitted together to obtain full length partners.

5. Upon positive partner identification, EST databases and general literature may be searched for information on gene expression ( i.e., mRNA, protein and activity levels)

a. in various tissues, cells, organisms;

b. in normal and pathologic states;

c. at various developmental times; and

d. other related or known proteins.

Based on partner identification the function of the expressed target gene may be postulated. Confirmation of its activity and function is then confirmed by detecting its activity in cells in which it is expressed.

### Computational Approach to Identify Natural Partner

After identification of a surrogate peptide binder, it is subjected to partner analysis using several different database search programs. In addition, the set of multiple surrogate peptide binders are aligned into groups based on motifs or consensus regions. Motifs and consensus regions can be identified by sequence alignment programs like MEME (Multiple EM for Motif Elicitation),

(http://meme.sdsc.edu/meme/website/intro.html). The motifs and consensus regions can be used as query patterns to search the available databases using MAST (Motif Alignment and Search Tool,

http://meme.sdsc.edu/mem/website/mast-intro.html) or Patternfind. The identified sequences can be further examined for significant differences in the expected frequency of amino acids and the number of time a specific peptide sequence has been repeated.

An example of a strategy for the computational approach to identifying a natural partner is shown below:

In the initial step, the entire peptide sequence and consensus motifs (if found) are entered into an Advanced BLAST search

(http://www.ncbi.nlm.nih.gov/blast/blast.cgi?Jform=1): using the following parameters:

Programs: blastp, tblastn

Databases: protein and nucleotide databases including dbest (ESTs), dsts (STSs) and htgs (unfinished high throughput genomic sequences)

Expect value: 1000 or 10000-20000

Matrix: PAM30 or PAM70

Query: Consensus motif alone and varying combinations of sequence at N- and C-terminal ends

In subsequent steps, motifs and consensus regions identified by sequence alignment programs like MEME are used as query patterns to search the available databases using Patternfind.

For Patternfind, the following parameters are used:

Databases: Nonredundant, Swissprot, TREST and TRGEN

Limit: Between 10 and 5000

Query: Consensus motif alone and varying combinations of sequence at N-and C-terminal ends

Data obtained from the various searches are analyzed under the following conditions:

Analyze results of different searches independently and then together to look for similar classes of proteins (eg. nucleic acid binding proteins, kinases) that may emerge.

Identify some of the best matches that show up in more than one kind of search (eg. same protein/ORF picked up by BLAST searches using different parameters, or by both BLAST and Patternfind) and compare sequence of protein in this region with other peptide surrogates containing this motif.

Examine potential significance of protein interaction in the context of the cellular function of target.

The output from each search are analyzed for partner hits based on the following criteria:

1. Search gives an exact match of at least 5-7 amino acids or appearance of the partner in at least 50% of the top cohort of any one search, and/or the appearance of the same or related hits occurring in multiple searches.

2. Search matches an expected class of protein partners based on function, cellular location or tissue/disease distribution.

3. Candidate produces a phenotype change when added into the appropriate model system.

Preferably, the partner hit has at least two of the criteria described above. More preferably, the partner hit appears in at least 50% of the top cohort of any one search or appears (or a related sequence appears) in multiple search results. Even more preferably, the partner hit has an exact match of at least 5-7 amino acids. Criterion 2 addresses the biological relevance of a hit (e.g., distribution, disease indication, etc.), and criterion 3 relates to the biological activity of the surrogate and its ability to cause a phenotypic change in the appropriate test system.

The homology between the partner and surrogate can range from being scattered over a long stretch (for example 15-25 amino acids) to a perfect match within a short sequence (at least 5-8 amino acids).

The generation of surrogates using large random and diverse libraries is target independent and their utility for partner identification resides in the computational analysis of the identified peptide's sequence. For successful partner identification to be feasible, surrogates must exhibit either the natural linear or conformational surface properties complementary to the target under investigation. The complementary peptide surface is selected via a biological enrichment process (i.e., panning) which is based on preferential binding potency to the target protein. Since the preferred libraries for use with the invention contain totally random peptides ranging from 10 and up to about 50 amino acids in length (and more preferably 20 to 40 amino acids in length), there are no known restrictions on the amino acids that can be selected to create the surrogate's ‘complementary’ surface. Thus, the examples described herein relate to the utility of the surrogate approach for finding the cognate receptor for both protein and non-protein targets. In the case of the surrogates for both HCV-mRNA and TNFβ, it is clear that the large diversity and size of the original library was, in fact, critical to their successful isolation since libraries of <20 amino acids peptides would not have contained either the KcB7 peptide or the HCV-specific surrogates.

In addition to the data presented in the Examples, we have screened other targets using this approach. While the expected natural partners were found for most of the proteins, there were some instances where surrogates were generated but lacked partner information (e.g., IGF-1R, growth hormone receptor, etc.). There are several possible explanations for these results.

### Examples of Targets Panned and Partners Revealed by Surrogate Peptides

| | Natural Site | Partner Revealed | Phenotype(s) of |
|---|---|---|---|
| Target Panned | of Action | by Surrogate | Surrogate Peptides |
| Coagulation | ExtraCell | none | antagonist |
| FIX | | | |
| TNF-β | ExtraCell | TNFR1 | antagonist |
| GHR | PI.Membr. | none | antagonist |
| IgAR | PI.Membr. | IgA | Agonist and |
| | | | antagonist |
| IGF-1R | PI.Membr. | none | Agonist and |
| | | | antagonist |
| IR | PI.Membr. | none | Agonist and |
| | | | antagonist |
| TNFR2 | PI.Membr. | TNF | Antagonist |
| | | ligands | |
| TNFR1 | PI.Membr | none | antagonist |
| TRAIL receptor | P.Membr | none | ND |
| fasR | P.Membr | fasL | antagonist |
| PAB 1620 | IntraCell | p53 | NT |
| (anti-p53 | | | |
| antibody) | | | |
| MDM-2 | IntraCell | p53 | antagonist |
| mRNA targets | IntraCell | RNA | NT |
| | | binding motif | |
| mRNA HCV | IntraCell | eIF3 | NT |

This Table gives a list of the targets panned using the 20 mer and 40 mer random libraries. Column 2 lists the putative site of biological action for each target. Column 3 describes whether a natural partner was found using a surrogate peptide found from the panning. Column 4 describes the biological activity of each surrogate in the appropriate biological assay.

Legend: Extracell: Target expressed as an extracellular protein; PI.Membr: Target expressed as a plasma membrane protein; IntraCell: Target expressed intracellularly; TNFβ, Tumor Necrosis Factor β; IgAR, IgA receptor; GHR, Growth Hormone receptor; IGF-1R, Insulin-like Growth Factor-1 receptor; IR, Insulin receptor; TNFR2, Tumor Necrosis Factor Receptor-2 (p75); TNFR1, Tumor Necrosis Factor receptor-1 (p55); NT=Not Tested.

While the libraries used are large and diverse, it is probable that identification of a surrogate peptide with partner information is a rare event. With that in mind, it may require the isolation and sequencing of large numbers of clones (perhaps >500/target) in order to find the appropriate surrogate for partner identification. In addition, some targets may have complex or unusual protein:protein contact sites that preclude generation of a surrogate with partner information.

Surrogates have also been found to have the minimal structural content necessary to induce a pharmacological effect on any target in addition to their use in partner identification. Most surrogates have been shown to have either agonist or antagonist activity in the appropriate biochemical and/or biological models (see Table above). Surrogates have also been shown to subdivide large contact surfaces into smaller contact domains through which target activity can be modified. These attributes provide for surrogate use in phenotyping and validating novel genes whose functions are unknown and for which there exist no known partners. Surrogates can also be used to develop competitive Site Directed Assays (SDAs) for each essential sub-domain, thereby allowing their use in high throughput screening of large combinatorial libraries of small molecules. See U.S. Pat. No. 6,010,861. Most peptide surrogates isolated from these complex libraries by routine panning procedures bind to regulatory hot spots on varied targets. This non-random association between a surrogate and a target's “hotspot” (i.e., pharmacological active site) assures a high degree of probability that, once found, surrogates will have utility for the rapid development of SDAs capable of identifying small molecules of pharmacological importance.

### Selecting Expression Systems of Original Target Gene for Phenotyping with Surrogate

Two expression systems may be used to assess phenotypic changes resulting from binding of the gene product with the surrogate. In one method, cells which express the gene product are identified and used as a natural expression system.

Information from EST data bases (cDNA libraries used to isolate ESTs; and others) is searched for the distribution of expressed cellular and tissue mRNA ( data collected by Northern blot analysis or other methods including but not limited to expression of protein or activity, if available) encoding the gene product. To identify high expression systems, surrogates may be labelled (via biotin, FITC tags), and used to probe tissue sections, tissue culture cells and organisms by immunological or fluorescent detection such as Elisas and FACS.

Alternatively, if natural expression systems are unavailable, an expression system may be created by expressing the gene in cells using standard techniques. Because the activity of the gene product may be cell type dependent, it is desirable to express the gene in a plurality of cell types.

### Expression and Purification of Novel Protein Open Reading Frames

No single heterologous expression system is adequate to produce all protein sequences in high yield and as fully folded active entities. In order to maximize the chances of recovering an active protein, any unknown new sequence should be expressed in multiple expression systems. One method for accomplishing this would be to sequentially clone the desired protein into several expression vectors optimized for individual cell culture expression systems. Alternatively, commercially available systems have been developed to allow protein sequences to be cloned and expressed in several cell culture systems simultaneously. One such system, the pTriEx™-1 Multisystem Vector, is available from Novagen. In this system, the protein sequence to be expressed is cloned into a multisystem vector incorporating consecutive CAG, T7Iac and p10 promoters. These three promoters allow high level expression from the single vector in mamalian, E. coli and insect cells, respectively. The vector also incorporates HSV•Tag® and His•Tag® tags on the c-terminus of expressed proteins to facilitate immunochemical detection and affinity purification. Expression levels can be checked using anti-HSV antibodies, and the crude proteins can be purified to near homogeneity using metal affinity chromatography. The purified protein would be suitable for use in biopanning and surrogate characterization.

### Detecting Phenotypic Changes

To detect the activity of the gene, phenotype changes (morphorlogical, biochemical, immunological) are observed following contact of the surrogate to the cell, tissue or organism. The surrogate may be free or attached to a penetrating peptide sequence, as anti-target probe, in fashion similar to known methods used with anti-sense technology.

Phenotyping can be done in natural systems if the target/target interaction is related to an observable phenotype. Under these conditions there is no need to over-express the target in a model cell.

The overall strategy for determining the functions of a gene by detecting changes in phenotype may be summarized as follows:

I. Obtain Surrogate

a. Make gene product of unknown functions for panning

i. Obtain oligoribonucleotides of 5′ and 3′ untranslated mRNA domains'

ii. Obtain full length DNA and express open reading frame (ORF) protein and purify ORF protein product

b. Pan peptide libraries ( phage, bacterial, yeast, mammalian cell or in vitro/ribosomal display) against gene product such as, for example:

i. Untranslated 3′ and 5′ mRNA domains, or

ii. ORF encoded protein

c. Sequentially make nth generation mutated libraries based on panned surrogate's sequences until a limited number of consensus sequences is obtained.

II. Use Surrogate Sequences to

a. Search data bases of translated consensus sequences and identify potential partner protein and genes.

b. Synthesize (or recombinantly express a fusion) surrogate consensus peptides obtained in the 1^{st} to n^{th} generation pan of peptide displayed libraries either

i. linked to cellular uptake peptide leader ( such as antanopedia) or

ii. free (i.e., with terminal amino acids for solubility if needed)

III. Use Soluble Surrogates to Detect Changes in Phenotype Mediated Through Activation of the Gene Product by the Surrogate

a. Add surrogates to intact model cells and quantitate effect

b. Add surrogates to in vitro model system and quantitate effect

c. Add surrogates at various doses and produce graded phenotypic knockouts.

The following non-limiting examples illustrate various aspects and embodiments of the invention and should not be contrived as limiting the scope of the invention. All references cited herein are incorporated herein by reference in their entirety.

### EXAMPLES

### Example 1

### Design of 40-mer and 20-mer Random Peptide Libraries

DNA fragments coding for peptides containing 40 random amino acids were generated by a PCR approach using synthetic oligonucleotides. A 145 base oligonucleotide was synthesized containing the sequence (NNK)₄₀ where N=A, C, T, or G and K=G or T. See U.S. Pat. Nos. 6,143,531, 5,681,726 and 388, which are hereby incorporated by reference. This oligonucleotide was used as the template in PCR reactions along with two shorter oligonucleotide primers, both of which are biotinylated at their 5′ ends. The resulting 190 bp product was purified and concentrated (followed by digestion with Sfil and Notl). The resulting 150 bp fragment was purified and the phagemid pCANTAB5E (Pharmacia) was digested with Sfil and Notl. The digested DNA was resolved using a 1% agarose gel, excised and purified by QIAEX II treatment (Qiagen). The vector and insert were ligated overnight at 15° C. The ligation product was purified. Electrocompetent cells were prepared by harvesting cells from a culture broth with an OD of 0.5-0.7 U_{OD}. by centrifugation in a fixed rotor for 10 minutes at 950 g. The cells were washed three times with ice cold pure water. Electroporations were performed at 1500 V in an electroporation cuvette (0.1 mm gap; 0.5 ml volume) containing 12.5 ug DNA and 500 uL of E. coli strain TG1 electrocompetent cells. Immediately after the pulse, 12.5 ml of pre-warmed (42° C.) 2×YT medium containing 2% glucose (YT-G) was added and the transformants grown at 37° C. for one hour. Cell transformants were pooled, the volume measured and an aliquot plated onto 2×YT-G containing 100 μg/ml ampicillin (YT-AG) to determine the number of transformants. The diversity of the random 40-mer peptide cell library was found to be >1.6×10¹⁰. The phage library was produced by rescue of the cell library according to standard phage preparation protocols. See e.g., Carcamo, et al. Proc. Natl Acad Sci USA (1998) 95: 11146-11151. Phage titers were usually 4×10¹³CFU/ml.

Sequencing of randomly selected clones from the cell library indicated that about 54% of all clones were in-frame. The short FLAG sequence, DYKD, was included at the N-terminus as an immunoaffinity tag. In addition, the E-tag epitope (GAPVPYPDPLEPR) was engineered into the carboxy terminus of the peptide.

A second random phage library of 20-mer peptides was constructed using the same approach. The diversity of this cell library was found to be >1.1×10¹¹ clones and sequencing revealed 77% of the clones were in frame.

### Example 2

### Panning TNF-β

A standard method was used to coat and block all microtiter plates. The target was diluted to 1 mg/ml in 50 mM sodium carbonate buffer, pH 9.5. One hundred microliters of this solution was added to an appropriate number of wells in a 96-well microtiter plate (MaxiSorp plates, Nunc) and incubated overnight at 4° C. Wells were then blocked with MPBS (PBS containing 2% non fat milk) at room temperature for one hour.

Eight wells being used for each round of panning. The phage for the phage library were incubated with MPBS for 30 minutes at room temperature, then 100 μl was added to each well. For the first round, the input phage titer was 4×10¹³ cfu/ml. For rounds 2 and 3, the input phage titer was approximately 10¹¹ cfu/ml. Phage were allowed to bind for two to three hours at room temperature. The wells were then quickly washed 13 times with 300 μl/well of MPBS. Bound phage were eluted by incubation with 100 μl/well of 20 mM glycine-HCl, pH 2.2 for 30 seconds. The resulting solution was then neutralized with Tris-HCl, pH 8.0. Log phase TG1 cells were infected with the eluted phage by incubation at 37° C. for 1 hr. Helper phage (M13KO7) was then added (multiplicity of infection(MOI)=1 5) and cells incubated in the presence of 50 μg/ml ampicillin and 2% glucose for 1 hr at 37° C. with shaking at 250 rpm. Following infection, cells were pelleted, resuspended in the initial culture volume of 2×YT containing 50 μg/ml ampicillin and 50 μg/ml kanamycin and grown overnight at 37° C. with shaking at 225 rpm. Cells from the overnight culture were pelleted and supernatant containing phage was recovered. Phage was precipitated with 6% PEG 8000, 300 mM NaCl and chilled on ice for 1 hr. Precipitated phage was pelleted by centrifugation at 10,000×g for 30 min, then resuspended in PBS+1 mM MgCl₂(1/100 of the initial volume). The phage was used for the next round of panning.

For Elisa analysis of individual clones, colonies were picked and phage prepared as described above using helper phage, M13KO7. Microtiter wells were coated and blocked as described above. Wells were coated with either IGF-1 R or a control IgG MAb. Phage were added at 100 μl/well and incubated at room temperature for 2 hr. The phage solution was then removed, and the wells were washed three times with PBS at room temperature. Anti-M13 antibody conjugated to horseradish peroxidase (Pharmacia Biotech) was diluted 1:3000 in MPBS and added to each well (100 μl/well). Incubation was for another hour at room temperature, followed by PBS washes as described. Color was developed by addition of ABTS solution (100 μl/well; Boehringer). Plates were analyzed at 405 nm using a SpectraMax 340 plate reader (Molecular Devices) and SoftMax Pro software. Data points were averaged after subtraction of appropriate blanks. A clone was considered “positive” if the A₄₀₅ of the well was ≧2-fold over background.

An additional series of panning experiments were performed using the eluted phage from the first panning of TNF-β. This additional panning, a subtractive panning, was included to remove any peptides that cross-reacted with other members of the TNF family. In particular, the phage was subsequently panned against TNFR1, TNFR2 and TNF-α.

The panning experiments identified a surrogate peptide, KcB7, with the amino acid sequence RKEMGGGGGPGWSENLFQ. A Blastp search, using several different queries revealed TNFR1 which is the natural biological partner of TNFβ.

BLASTp search results for the TNFβ Surrogate peptide KcB7

| | |
|---|---|
| Query: | WSENLFQ |
| Database: | nr |

| | | |
|---|---|---|
| | Score E | |
| Sequences producing significant alignments: | (bits) | Value |
| prf\|\|2102238A tumor necrosis factor alpha inhibitor [Homo s. . . | 20 | 2419 |
| gb\|AAA36756.1\| (M60275) TNF receptor [Homo sapiens | 20 | 2419 |
| pdb\|1TNR\|R Chain R, Tumor Necrosis Factor Receptor P55. . . | 20 | 2419 |
| pdb\|1NCF\|A Chain A, Binding Protein, Cytokine Mol_id: 1; Mo. . . | 20 | 2419 |
| ref\|NP_001056.1\| tumor necrosis factor receptor 1 (55kD) >g. . . | 20 | 2419 |
| >prf\|\|2102238A tumor necrosis factor alpha inhibitor [Homo sapiens] | | |
| Length = 160 | | |
| Score = 20.4 bits (41), Expect = 2419 | | |
| Identities = 7/7 (100%), Positives = 7/7 (100%) | | |

| | | | |
|---|---|---|---|
| Query: | 1 | WSENLFQ | 7 |
| | | WSENLFQ | |
| Sbjct: | 96 | WSENLFQ | 102 |

>gb|AAA36756.1| (M60275) TNF receptor [Homo sapiens]
Length = 453
Score = 20.4 bits (41), Expect 2419
Identities = 7/7 (100%), Positives = 7/7 (100%)

| | | | |
|---|---|---|---|
| Query: | 1 | WSENLFQ | 7 |
| | | WSENLFQ | |
| Sbjct: | 136 | WSENLFQ | 142 |

22 pdb|1TNR|R Chain R, Tumor Necrosis Factor Receptor P55 (Extracellular
Domain) Complexed With Tumor Necrosis Factor-Beta
Length = 139
Score = 20.4 bits (41), Expect = 2419
Identities = 7/7 (100%), Positives = 7/7 (100%)

Patternfind search results for the TNF-β Surrogate peptide KcB7

| | |
|---|---|
| Query sequence: | WSENLFQ |
| IV. DATABASE: | NONREDUNDANT |
| Limit | 10 |

gp|M60275|339760|AC886035F969E231 TNF receptor [Homo sapiens]

| | | |
|---|---|---|
| Occurences: | 1 | |
| Position: | 136 | WSENLFQ |

sp|P19438|TNR1_HUMAN|4CEFBA96D03B8225 (TNFRSF1A. . . )TUMOR NECROSIS
FACTOR RECEPTOR 1 PRECURSOR (TUMOR NECROSIS FACTOR BINDING
PROTEIN 1) (TBPI) (P60) (TNF-R1) (TNF-RI) (P55) (CD120A).[Homo sapiens]

| | | |
|---|---|---|
| Occurences: | 1 | |
| Position: | 136 | WSENLFQ |

2 matches found

Closer examination of the complementary sequences revealed that the short N-terminal sequence RKEMG and the C-terminal sequence WSENLFQ were identical to regions on TNFR1 (amino acids 77-81 and 107-113 respectively). These segments corresponded to amino acids within two critical ligand:receptor contact domains. In the case of the N-terminal grouping, the surrogate contained 5 of the 15 amino acids of the 77-81 contact domain whereas in the C-terminal grouping, the surrogate contained 6 of the 9 amino acids identified within the 107-113 contact domain.

Comparison with human TNFRI extracellular domain
IYPSGVJGLVPHLGDREKRDSVCPQGKYJHPQNNSICCTKCHKGTYLYNPCPGPG
QDTDCRECesgsFTASENHLRhcLscSkCRkeMgQVEISSCTVDRDTVCGCRKNQYR
HYWSENLFgcFNCSLCLNGTVHLSCQEKQNTVCTCHAGFFLRENECVSCS
Contact residues are based on Banner et al., (1993) Cell 73: 431-445.
Bold = contacted by TNFβ subunit A
lower case = contacted by TNFβ subunit C
italics =contacted by TNFβ both subunits A and C
Underline = homology to the φ clone
TNFβ
LPGVGLTPSAAQTARQHPKMHLAHSTLKPAAHLIGDPSKQNSLLWRANTDRAF
LQDGFSLSNNSLLVPTSGIYFVYSQVVFSGKAYSPKAPSspLyLAHEVQLFSsqypfH
vPLLSSqKmVYPGLQEPWLHSMYHGAAFQLTQGDQLSThTdGIPHLVLSPSTVFF
GAFAL
Bold = TNFβ subunit A
lower case = TNFβ subunit C
—
Comparison with human TNFR2 extracellular domain
rKEMGGGGGGpgwSEN1FQ
LPAQVAFTPYAPEPGSTCRLREYYDQTAQMCCSKCSPGQHAKVFCTKTSDTVCD
SCEDSTYTQLWNWVPECLSCGSRCSSDQVETQACTrEQNRJCTCRpgwYCA1SKQ
EGCRLCAPLRKCRPGFGVARPGTETSDVVCKPCAPGTFSNTTSSTDJCRPHQICN
VVAIPGNASRDAVCTSTSPT

### EXAMPLE 3

### Panning RNA Target

Surrogate peptides were obtained by panning a portion of the 5′ UTR of HCV mRNA using both the 20 mer and 40 mer random libraries. All solutions and surfaces were pretreated with DEPC or RNaseZap (Ambion, Inc.), respectively, to eliminate RNase contamination that may compromise the integrity of the RNA. Biotinylated—RNA target was diluted to 1 mg/ml in binding buffer (PBS containing 1 mM MgCl₂), denatured at 65° C. for 5 min and reannealed by slow cooling to room temperature to allow for appropriate refolding. The synthetic biotinylated-RNA target had the following sequence, 5′-biotin′AA UUG CCA GGA CGA CCG GGU CCU UUC UUG GAU CM CCC GCU CM UGC CUG GAG AUU-3′. Reannealed RNAs were stored in small aliquots (10-25 μl/tube) at −20° C. Microtiter wells were treated with RNaseZap (Ambion, Inc.) before use. One hundred microliters of RNA solution diluted to 2.5 ng/μl was added to an appropriate number of wells in a 96-well microtiter plate precoated with Streptavidin (Pierce) and incubated for 1 hr at room temperature. Unbound streptavidin was then blocked with 50 μl of 2 mM biotin at room temperature for 1 hr. Four wells were used for each round of panning and 100 μl phage was added to each well. Pahge were precipitated with RNase-free 6% PEG 8000+0.3 M NaCl, washed with the same solution once and resuspended in RNase-free PBS+1 mM MgCl₂+Superasin (RNase inhibitor from Ambion, Inc.). For the first round, the input phage titer was 1×10¹³ cfu/ml. For rounds 2 and 3, the input phage titer was approximately 10¹¹ cfu/ml. Phage were allowed to bind for two to three hours at room temperature. The wells were then quickly washed 13 times with 400 μl/well of PBS. Bound phage were eluted by incubation with 150 μl/well of 50 mM glycine-HCl, pH 2.2+0.1% BSA for 5 min. The resulting solution was then neutralized with Tris-HCl, pH 8.0. Log phase TG1 cells were infected with the eluted phage by incubation at 37° C. for 1 hr. Helper phage (M13KO7) was then added (multiplicity of infection(MOI)=15) and cells incubated in the presence of 50 μg/ml ampicillin and 2% glucose for 1 hr at 37° C. with shaking at 250 rpm. Following infection, cells were pelleted, resuspended in the initial culture volume of 2×YT containing 50 μg/ml ampicillin and 50 μg/ml Kanamycin and grown overnight at 37° C. with shaking at 225 rpm. Cells from the overnight culture were pelleted and supernatant containing phage was recovered. Phage was precipitated with 6% PEG 8000, 300 mM NaCl and chilled on ice for 1 hr. Precipitated phage was pelleted by centrifugation at 10,000×g for 30 min, washed once with the same solution and then resuspended in PBS+1 mM MgCl₂ (1/100 of the initial volume).

For Elisa analysis of individual clones, colonies were picked and phage prepared as described above using helper phage, M13KO7. Streptavidin-coated microtiter plates were blocked with PBS containing 2% non fat milk for 1 hr at room temperature, treated with RNaseZap, then coated with biotinylated RNA target (100 ng/well) by incubation for 1 hr at room temperature. Superasin (RNase inhibitor from Ambion, Inc.) was added to the wells prior to addition of 100 μl/well of phage from isolated clones and incubated at room temperature for 2 hr. The phage solution was then removed, and the wells were washed three times with PBS at room temperature. Anti-M13 antibody conjugated to horseradish peroxidase (Pharmacia Biotech) was diluted 1:3000 in PBS (also containing Superasin) and added to each well (100 μl/well). Incubation was for another hour at room temperature, followed by PBS washes as described. Color was developed by addition of ABTS solution (100 μl/well; Boehringer). Plates were analyzed at 405 nm using a SpectraMax 340 plate reader (Molecular Devices) and SoftMax Pro software. Data points were averaged after subtraction of appropriate blanks. A clone was considered “positive” if the A₄₀₅ of the well was >2-fold over background.

Peptides HCV-3-F5, HCV-3-H8 and HCV-NG-D9 were obtained from the 40-mer library. Peptide HCV-3-C3 was obtained from the 20 mer library. Sequence analysis of these surrogate peptide binders to HCV using MEME (Motif Elicitation Program) and other peptide sequence alignment programs identified a consensus sequence TxRLL. Database searches using BLAST and Patternfind identified a human gene product, subunit p170 of elF3. The consensus sequences are shown below in bold and underlined. Sequences outside the motif that are conserved between the surrogates and elF3 are in Italics and underlined.

HCV ALIGNMENTS

eIF3: EDLDNIQTPE-SVLLSAVSGEDTQDRTDRLLLTPWVKFLWESY
CONSENSUS: TxRLL
HCV-NG-D9 TSGESSGDRTRRVLTSSSARTILPN
HCV-3-F5 LLVTGQFP--SQLLLGGAVCGP--STPRLRTGLCRLSGT
HCV-3-H8 RRTCGDPAAMLERLSCRAGDYRGASHTGRLLNLRGMHQYP
HCV-3-C3 FTTPRHLSGRTVQMMRDSTS

OUTPUT FROM ADVANCED BLAST SEARCH FOR HCV mRNA
SURROGATE QUERY − SEARCH 1:

Query sequence: TSGESSGDRTRRVLT
Program: blastp
Database: swissprot
Expect value: 10000

OUTPUT:

| | | |
|---|---|---|
| | Score | E |
| Sequences producing significant alignments: | (bits) | Value |

| | | |
|---|---|---|
| sp\|P31258\|HXAB_CHICK HOMEOBOX PROTEIN HOX-A11 (GHOX-1I) (CH... | 20 | 477 |
| sp\|P23116\|IF3A_MOUSE EUKARYOTIC TRANSLATION INITIATION FACT... | 19 | 1072 |
| sp\|P39690\|KHS1_YEAST KILLER TOXIN KES PRECURSOR (KILLER OF ... | 19 | 1072 |
| sp\|083264\|NUSG_TREPA TRANSCRIPTION ANTITERIVIINATION PROTEIN ... | 19 | 1072 |
| sp\|P13079\|CARB_STRTH RRNA METHYLTRANSFERASE | 19 | 1072 |
| (CARBOMYCIN-RES... | | |
| sp\|Q14152\|IF3A_HUMAN EUKARYOTIC TRANSLATION INITIATION FACT... | 19 | 1072 |
| sp\|P39925\|AFG3_YEAST MITOCHONDRIAL RESPIRATORY CHAIN COMPLE... | 19 | 1072 |
| sp\|P16561\|HEMA_VACCT HEMAGGLUTININ PRECURSOR | 19 | 1404 |
| sp\|P52023\|DP3B_SYNP7 DNA POLYMERASE III, BETA CHAIN | 19 | 1404 |
| sp\|P20978\|HEMA_VACCC HEMAGGLUTININ PRECURSOR | 19 | 1404 |
| sp\|P15989\|CA36_CHICK COLLAGEN ALPHA 3(VI) CHAIN PRECURSOR | 19 | 1404 |
| List truncated here... | | |
| >sp\|P31258\|HXAB_CHICK HOMEOBOX PROTEIN HOX-A11 (GHOX-1I) | | |
| (CHOX-1.9) | | |
| Length = 297 | | |
| Score = 20.4 bits (41), Expect = 477 | | |
| Identities = 8/11 (72%), Positives = 9/11 (81%) | | |
| Query: 2 SGESSGDRTRR 12 | | |
| SG SSG RTR+ | | |
| Sbjct: 217 SGSSSGQRTRK 227 | | |
| >sp\|P23116\|IF3A_MOUSE EUKARYOTIC TRANSLATION INITIATION | | |
| FACTOR 3 SUBUNIT 10 (EIF-3 THETA) | | |
| (EIF3 P167) (EIF3 P180) (EIF3 P185) (P162 | | |
| PROTEIN) | | |
| (CENTROSOMIN) | | |
| Length = 1344 | | |
| Score = 19.2 bits (38), Expect = 1072 | | |
| Identities = 8/13 (61%), Positives = 10/13 (76%) | | |
| Query: 2 SGESSGDRTRRVL 14 | | |
| SGE + DRT R+L | | |
| Sbjct: 133 SGEDTQDRTDRLL 145 | | |
| >sP\|P396901KHS1_YEAST KILLER TOXIN KHS PRECURSOR (KILLER OF | | |
| HEAT SENSITIVE) | | |
| Length = 708 | | |
| Score = 19.2 bits (38), Expect = 1072 | | |
| Identities = 8/13 (61%), Positives = 10/13 (76%) | | |
| Query: 3 GESSGDRTRRVLT 15 | | |
| G+SSG T+R LT | | |
| Sbjct: 98 GKSSGSATKRGLT 110 | | |
| >sp\|083264\|NUSG_TREPA TRANSCRIPTION ANTITERMINATION PROTEIN | | |
| NUSG | | |
| Length = 185 | | |
| Score = 19.2 bits (38), Expect = 1072 | | |
| Identities = 7/12 (58%), Positives = 9/12 (74%) | | |
| Query: 2 SGESSGDRTRRV 13 | | |
| +GE GDRT R+ | | |
| Sbjct: 117 AGEIKGDRTPRI 128 | | |
| >sp\|P13079\|CARB_STRTH RRNA METHYLTRANSFERASE (CARBOMYCIN- | | |
| RESISTANCE PROTEIN) | | |
| Length = 299 | | |
| Score = 19.2 bits (38), Expect = 1072 | | |
| Identities = 8/12 (66%), Positives 8/12 (66%) | | |
| Query: 2 SGESSGDRTRRV 13 | | |
| SG S DR RRV | | |
| Sbjct: 40 SGRSEADRRRRV 51 | | |
| >sp\|Q14152\|IF3A_HUMAN EUKARYOTIC TRANSLATION INITIATION | | |
| FACTOR 3 SUBUNIT 10 (EIF-3 THETA) | | |
| (EIF3 P167) (EIF3 P180) (EIF3 P185) (KIAA0139) | | |
| Length = 1382 | | |
| Score = 19.2 bits (38), Expect = 1072 | | |
| Identities = 8/13 (61%), Positives = 10/13 (76%) | | |
| Query: 2 SGESSGDRTRRVL 14 | | |
| SGE + DRT R+L | | |
| Sbjct: 133 SGEDTQDRTDRLL 145 | | |
| >sp\|P39925\|AFG3_YEAST MITOCHONDRIAL RESPIRATORY CHAIN | | |
| COMPLEXES ASSEMBLY PROTEIN AFG3 | | |
| (TAT-BINDING HOMOLOG 10) | | |
| Length = 761 | | |
| Score = 19.2 bits (38), Expect = 1072 | | |
| Identities = 8/14 (57%), Positives = 10/14 (71%) | | |
| Query: 2 SGESSGDRTRRVLT 15 | | |
| S +SGD + RVLT | | |
| Sbjct: 136 SSNNSGDDSNRVLT 149 | | |

OUTPUT FROM ADVANCED BLAST SEARCH FOR HCV mRNA
SURROGATE QUERY - SEARCH 2:

Query sequence: TSGESSGDRTRRVLTSSS
Program: blastp
Database: swissprot
Expect value: −e 10000

| | | |
|---|---|---|
| | Score | E |
| Sequences producing significant alignments: | (bits) | Value |
| sp\|Q01728\|NAC1_RAT SODIUM/CALCIUM EXCHANGER 1 PRECURSOR (NA... | 21 | 65 |
| sp\|P70414\|NAC1_MOUSE SODIUM/CALCIUM EXCHANGER 1 PRECURSOR (... | 21 | 65 |
| sp\|P48765\|NAC1_BOVIN SODIUM/CALCIUM EXCHANGER 1 PRECURSOR (... | 20 | 190 |
| sp\|P48766\|NAC1_CAVPO SODIUM/CALCIUM EXCHANGER 1 PRECURSOR (... | 20 | 190 |
| sp\|P32418\|NAC1_HUMAN SODIUM/CALCIUM EXCHANGER 1 PRECURSOR (... | 20 | 190 |
| sp\|P23685\|NAC1_CANFA SODIUM/CALCIUM EXCHANGER 1 PRECURSOR (... | 20 | 190 |
| sp\|P48767\|NAC1_FELCA SODIUM/CALCIUM EXCHANGER 1 PRECURSOR (... | 20 | 190 |
| sp\|P08173\|ACM4_HUMAN MUSCARINIC ACETYLCHOLINE RECEPTOR M4 | 19 | 249 |
| sp\|P23116\|IF3A_MOUSE EUKARYOTIC TRANSLATION INITIATION FACT... | 19 | 249 |
| sp\|Q14152\|IF3A_HUMAN EUKARYOTIC TRANSLATION INITIATION FACT... | 19 | 249 |
| sp\|P15656\|FGF5_MOUSE FIBROBLAST GROWTH FACTOR-S PRECURSOR (... | 19 | 327 |
| sp\|P30042\|ES1_HUMAN ES1 PROTEIN HOMOLOG PRECURSOR (PROTEIN ... | 19 | 327 |
| sp\|O35491\|CLK2_MOUSE PROTEIN KINASE CLK2 | 18 | 428 |
| sp\|P49760\|CLK2_HUMAN PROTEIN KINASE CLK2 | 18 | 428 |
| sp\|P15172\|MYOD_HUMAN MYOBLAST DETERMINATION PROTEIN 1 (MYOG... | 18 | 428 |
| sp\|075069\|Y481_HUMAN HYPOTHETICAL PROTEIN KIAA0481 (HH1480) | 18 | 428 |
| sp\|P02533\|K1CN_HUMAN KERATIN, TYPE I CYTOSKELETAL 14 (CYTOK... | 18 | 561 |
| sp\|P30989\|NTR1_HUMAN NEUROTENSIN RECEPTOR TYPE 1 (NT-R-1) (... | 18 | 561 |
| sp\|P30551\|CCKR_RAT CHOLECYSTOKININ TYPE A RECEPTOR (CCK-A R... | 18 | 561 |
| sp\|Q08369\|GAT4_MOUSE TRANSCRIPTION FACTOR GATA-4 (GATA BIND... | 18 | 561 |
| List truncated here... | | |
| >sp\|Q0l728\|NAC1_RAT SODIUM/CALCIUM EXCHANGER 1 PRECURSOR | | |
| (NA+/CA2+−EXCHANGE PROTEIN 1) | | |
| Length = 971 | | |
| Score = 21.2 bits (43), Expect = 65 | | |
| Identities = 9/15 (60%), Positives = 11/15 (73%) | | |
| Query: 3 GESSGDRTRRVLTSS 17 | | |
| GE G RT ++LTSS | | |
| Sbjct: 933 GELGGPRTAKLLTSS 947 | | |
| >sp\|P704l4\|NAC1_MOUSE SODIUM/CALCIUM EXCHANGER 1 PRECURSOR | | |
| (NA+/CA2+−EXCHANGE PROTEIN 1) | | |
| Length = 970 | | |
| Score = 21.2 bits (43), Expect = 65 | | |
| Identities = 9/15 (60%), Positives = 11/15 (73%) | | |
| Query: 3 GESSGDRTRRVLTSS 17 | | |
| GE C RT ++LTSS | | |
| Sbjct: 932 GELGGPRTAKLLTSS 946 | | |
| >sp\|P48765\|NAC1_BOVIN SODIUM/CALCIUM EXCHANGER 1 PRECURSOR | | |
| (NA+/CA2+−EXCHANGE PROTEIN 1) | | |
| Length = 970 | | |
| Score = 19.6 bits (39), Expect = 190 | | |
| Identities = 8/14 (57%), Positives = 10/14 (71%) | | |
| Query: 3 GESSGDRTRRVLTS 16 | | |
| GE G RT ++LTS | | |
| Sbjct: 932 GELGGPRTAKLLTS 945 | | |
| >sp\|P48766\|NAC1_CAVPO SODIUM/CALCIUM EXCHANGER 1 PRECURSOR | | |
| (NA+/CA2+−EXCHANGE PROTEIN 1) | | |
| Length = 970 | | |
| Score = 19.6 bits (39), Expect = 190 | | |
| Identities = 8/14 (57%), Positives 10/14 (71%) | | |
| Query: 3 GESSGDRTRRVLTS 16 | | |
| GE G RT ++LTS | | |
| Sbjct: 932 GELGGPRTAKLLTS 945 | | |
| >sp\|P32418\|NAC1_HUMAN SODIUM/CALCIUM EXCHANGER 1 PRECURSOR | | |
| (NA+/CA2+−EXCHANGE PROTEIN 1) | | |
| Length = 970 | | |
| Score = 19.6 bits (39), Expect = 190 | | |
| Identities = 8/14 (57%), Positives = 10/14 (71%) | | |
| Query: 3 GESSGDRTRRVLTS 16 | | |
| GE G RT ++LTS | | |
| Sbjct: 932 GELGGPRTAKLLTS 945 | | |
| >sP\|P23685\|NAC1_CANFA SODIUM/CALCIUM EXCHANGER 1 PRECURSOR | | |
| (NA+/CA2+−EXCHANGE PROTEIN 1) | | |
| Length = 970 | | |
| Score = 19.6 bits (39), Expect = 190 | | |
| Identities = 8/14 (57%) 1 Positives = 10/14 (71%) | | |
| Query: 3 GESSGDRTRRVLTS 16 | | |
| GE G RT ++LTS | | |
| Sbjct: 932 GELGGPRTAKLLTS 945 | | |
| >sp\|P48767\|NACl_FELCA SODIUM/CALCIUM EXCHANGER 1 PRECURSOR | | |
| (NA+/CA2+−EXCHANGE PROTEIN 1) | | |
| Length = 970 | | |
| Score = 19.6 bits (39), Expect = 190 | | |
| Identities = 8/14 (57%), Positives = 10/14 (71%) | | |
| Query: 3 GESSGDRTRRVLTS 16 | | |
| GE G RT ++LTS | | |
| Sbjct: 932 GELGGPRTAKLLTS 945 | | |
| >sp\|P08173\|ACM4_HUMAN MUSCARINIC ACETYLCHOLINE RECEPTOR M4 | | |
| Length = 479 | | |
| Score = 19.2 bits (38), Expect = 249 | | |
| Identities = 8/14 (57%), Positives = 13/14 (92%) | | |
| Query: 5 SSGDRTRRVLTSSS 18 | | |
| SSG+++R++TSSS | | |
| Sbjct: 10 SSGNQSVRLVTSSS 23 | | |
| >sp\|P23116\|IF3A_MOUSE EUKARYOTIC TRANSLATION INITIATION | | |
| FACTOR 3 SUBUNIT 10 (EIF-3 THETA) | | |
| (EIF3 P167) (EIF3 P180) (EIF3 P185) (P162 | | |
| PROTEIN) | | |
| (CENTROSOMIN) | | |
| Length = 1344 | | |
| Score = 19.2 bits (38), Expect = 249 | | |
| Identities = 8/13 (61%), Positives = 10/13 (76%) | | |
| Query: 2 SGESSGDRTRRVL 14 | | |
| SGE + DRT R+L | | |
| Sbjct: 133 SGEDTQDRTDRLL 145 | | |
| >sP\|Q14152\|IF3A_HUMAN EUKARYOTIC TRANSLATION INITIATION | | |
| FACTOR 3 SUBUNIT 10 (EIF-3 THETA) | | |
| (EIF3 P167) (EIF3 P180) (EIF3 P185) (KIAA0139) | | |
| Length = 1382 | | |
| Score = 19.2 bits (38), Expect = 249 | | |
| Identities = 8/13 (61%), Positives = 10/13 (76%) | | |
| Query: 2 SGESSGDRTRRVL 14 | | |
| SGE + DRT R+L | | |
| Sbjct: 133 SGEDTQDRTDRLL 145 | | |
| >sp\|P15656\|FGF5_MOUSE FIBROBLAST GROWTH FACTOR-S PRECURSOR | | |
| (FGF-5) (HBGF-5) | | |
| Length = 264 | | |
| Score = 18.8 bits (37), Expect = 327 | | |
| Identities = 9/16 (56%), Positives = 10/16 (62%) | | |
| Query: 3 GESSGDRTRRVLTSSS 18 | | |
| G+SSG R R TSS | | |
| Sbjct: 39 GDSSGSRGRSSATFSS 54 | | |
| >sp\|P30042\|ES1_HUMAN ES1 PROTEIN HOMOLOG PRECURSOR (PROTEIN | | |
| KNP-1) (GT335) | | |
| Length = 268 | | |
| Score = 18.8 bits (37), Expect = 327 | | |
| Identities = 8/18 (44%), Positives = 11/18 (60%) | | |
| Query: 1 TSGESSGDRTRRVLTSSS 18 | | |
| T G+ S +R VLT S+ | | |
| Sbjct: 93 TKGQPSEGESRNVLTESA 110 | | |
| >sp\|035491\|CLK2_MOUSE PROTEIN KINASE CLK2 | | |
| Length = 499 | | |
| Score = 18.4 bits (36), Expect = 428 | | |
| Identities = 8/11 (72%), Positives = 8/11 (72%) | | |
| Query: 2 SGESSGDRTRR 12 | | |
| S SS DRTRR | | |
| Sbjct: 34 SWSSSSDRTRR 44 | | |
| >sp\|P49760\|CLK2_HUMAN PROTEIN KINASE CLK2 | | |
| Length = 499 | | |
| Score = 18.4 bits (36), Expect = 428 | | |
| Identities = 8/11 (72%), Positives = 8/11 (72%) | | |
| Query: 2 SGESSGDRTRR 12 | | |
| S SS DRTRR | | |
| Sbjct: 34 SWSSSSDRTRR 44 | | |

Database searches using Patternfind at the ISREC server were performed using parameters appropriate for short protein queries and were successful in identifying a human gene product, subunit p170 of elF3. Searches using the consensus region as the query likewise identified sequence homology to the large subunit p170 of elF3.

Output from Patternfind for HCV mRNA surrogate query

Query sequence: DRTXRLL
Database: Nonredundant
Limit: 10
sp|Q14152|IF3A_HUMAN|485C01B28D67EBBA (EIF3S10)EUKARYOTIC
TRANSLATION INITIATION FACTOR 3 SUBUNIT 10 (EIF-3 THETA) (EIF3 P167)
(EIF3 P180) (EIF3 P185) (KIAA0139).[Homo sapiens]
Occurrences: 1
Position: 139 DRTDRLL
sp|P4637 3|FAS1_RHOFA|A66B6F3DF1286566 (FAS1..)CYTOCHROME P450 FAS 1 (EC 1.14.-.-).
[Rhodococcus fascians]
Occurrences: 1
Position: 170 DRTARLL
sp|P23116|IF3A_MOUSE|F4CAE2169F577712 (EIF3S10..)EUKARYOTIC
TRANSLATION INITIATION FACTOR 3 SUBUNIT 10 (EIF-3 THETA) (EIF3 P167)
(EIF3 P180) (EIF3 P185) (P162 PROTEIN) (CENTROSOMIN).[Mus musculus]
Occurrences: 1
Position: 139 DRTDRLL

### EXAMPLE 4:

### Panning mRNA

Short linear amino acid domains found in naturally occurring RNA-binding proteins were identified in peptides isolated from the random peptide libraries. These domains are generic, i.e. general RNA binding protein motifs rather than specific RNA binding motifs. Surrogate peptides were obtained by panning a portion of the 5′ UTR of four different mRNA targets using both the 20 mer and 40 mer random libraries as described in Example 3. Isolated phage binders from rounds three and four of each pan were sequenced. For each mRNA target, the predicted amino acid sequences of the peptide binders were analyzed for both overall amino acid content and the occurrence of known RNA-binding motifs and consensus domains. All of the peptide binders showed enrichment of arginine residues, as would be expected for RNA binding proteins. Also, tryptophan, serine, and glycine residues were enriched. The following table gives a comparison of the specific amino acid composition of peptide binders with regard to their average frequency of occurrence seen within the original unpanned library. These data were compared to the actual frequency of occurrence in the library before and after panning on the various mRNA targets denoted as M1, M2, and M3. All numbers are expressed as a percentage of the expected frequency.

| | Arg | Gly | Trp | Ser |
|---|---|---|---|---|
| Exp. Freq. | 9.4 | 6.3 | 3.1 | 9.4 |
| Library | 9.4 | 11.6 | 3.1 | 7.7 |
| M1 | 13.9 | 12.6 | 5.2 | 10.0 |
| M2 | 13.0 | 13.0 | 4.3 | 8.3 |
| M3 | 12.4 | 12.3 | 5.1 | 9.8 |

In addition, several peptides from each pan showed the presence of the RGG box, a well-defined RNA-binding motif, as indicated below. RGG sequences in each surrogate is in bold and underlined.

| | |
|---|---|
| M1-3-B7 | RGLFTEWFRGGSWSNYRVTS |
| M1-3-E8 | TDGGRSVISDNVRGGSRLWLWIRHGSWSQAWGPQDAWSSK |
| M1-3-H6 | RVSSAQPGCTSRVRFRCPRGGLLFNGVTSTNPKTGLSNAQ |
| M1-4-H1 | VVYVGVLSYWPHLSGGGRLQVRCLIGRGGFGCRGG |
| M2-3-C1 | WPPGRTLSDLIRGGAGARGM |
| M2-3-C9 | SSGGLHRWSALRGGHGHGLA |
| M2-3-E2 | AMRLKPIAFKGPRAGAGWVEVQPCFAAFRAACTRGGSHHH |
| M2-3-E3 | LHAGWDVTAPRRACKGAQGPGLHGRFYCHRGGLCSGLGRC |
| M2-3-E9 | DEQSSLKGKLRGALVRLGMGHAMPHRGGVWPSTGRPSKQG |
| M2-3-H12 | WTPRHGPMRCWRHQSVFPVGAGPHWALWPIKGPRGGRTAC |
| M2-NG-C7 | RKTGSNIWLPLYHKVCPASTRAGNGRGGSRFLWGSMQTNC |
| M3-3-B9 | RLQRRGGGAVAVVWVGFGVGLLWGRLLLIILGWVLMWFLS |
| M3-3-C2 | QHSEHGGTEWRKRGGMAFAASFLCMRDSYRTTRLRSLLG |
| M3-3-C7 | GTRHVINRVRDSSGVPCKRFGGLQFSQMGKCTIPRGGA |
| M4-NG-A4 | VLRGGSVGKGSLMWCQEVDWRTGGPRSNLWGLWNGRQPPK |

Furthermore, one sequence was found from panning the 20-mer random peptide library on traget M1 that contained the KH motif, which is also a known RNA-binding motifs. The surrogate motif corresponding to the KH domain is in bold and underlined.

| | |
|---|---|
| KH Motif | VIGxxGxxF |
| M1-3-C6 | GVIGGRGLLFPLSGFLHQHR |

### EXAMPLE 5

### Panning of Tie-1(Pro-angiogenic Tyrosine Kinase)

Surrogates acting as for Tie-1 were identified by panning against Tie-1. Six wells of a 96-well microtiter plates were coated with Tie-1 extracellular domain (R&D Systems) at concentrations ranging from 50-500 ng/well). Plates are incubated overnight at 4° C. At the same time, an aliquot of E. coli, strain TG1 was inoculated into 2×YT media and grown overnight at 37° C. The next day, unbound antigen was removed and the coated wells were blocked with 300 ul of 2% non-fat milk in PBS (NFM-PBS) for one hour at room temperature. The plates were then washed plates 3 times with PBS. The phage libraries were thawed and mixed with 0.1 vol of PBS-2% non-fat milk (NFM), 100 μl of each library was added to the antigen-coated wells and the plates are incubated for 3 hours at room temperature. Each well was washed 13 times with PBS-2% NFM and the phage eluted with 100 ul of 50 mM glycine-HCL containing 0.1% BSA (pH2.2) following a five minute incubation. The eluted phage from each library was pooled, neutralized with 100 ul of 1 M Tris-HCl (pH 8.0), and added to 10 ml of log phase E coli TG1 (OD₆₀₀=1.0), and amplified in 2×YT- glucose medium for one hour at 37° C. Helper phage (M13K07) and ampicillin were then added and the cells were incubated for an additional hour at 37° C. The cells were pelleted at 3500 RPM for 20 minutes, resuspended in 2×YT-AK medium (YT medium containing ampicillin and kanamycin) and incubated overnight at 37° C. The next day, the infected bacterial cells were centrifuged at 3500 RPM at 40C for 15 minutes and the pellet discarded. The supernatant contained the phage and was precipitated with ¼ volume of 30% PEG-8000 in 1.6 M NaCl by incubating on ice for 1 hour. The precipitant was centrifuged at 10,000 RPM at 4° C. for 30 minutes and the phage pellet resupended in about 1 ml of NFM-PBS. The phage was then used for the next round of panning. Three-four rounds of panning were done for both the 20-mer and 40-mer libraries. Two to three hundred random clones were picked from rounds 3 and 4 and grown in 96 well cluster plates as a master stock.

For screening, 40 ul of master stock was transferred from each master to another set of cluster tubes containing 400 μl of 2×YT-AG and helper phage (final concentration of 5×10¹⁰/ml). The tubes were incubated at 37° C. with constant shaking for two hours. The cultures were centrifuged at 2500×g at 4° C. for 20 minutes, the supernatant was discarded, and the bacterial pellet was resuspended in 400 ul of 2×YT-AK medium and was incubated overnight at 37° C. At that time, the cells were removed by centrifugation at 2500×g and the supernatants were transferred to a new set of cluster tubes and used in ELISA or stored at 4° C.

Each well of a MaxiSorp plate (Nunc) was coated with 100 μl of target (1 μg/ml) overnight at 4° C. The wells were blocked with NFM-PBS for 1.5 hours at room temperature. Phage was added at 100 ul/well and the plates incubated for 3 hours at room temperature. After washing 3× with PBS-Tween, plates were probed with an anti-M13 antibody conjugated to horseradish peroxidase (1:3000 in PBS-NFM) for 1 hour at room temperature followed by addition of 100 ul of ABTS for 15-30 minutes at room temperature. The OD was measured using a SpectraMax Microplate Spectrophotometer (Molecular Devices) at 405 nM after a 30 minute incubation at room temperature.

A total of 104 binders were sequenced yielding 32 unique sequences. Several different peptide motifs were identified that selectively bind to the Tie-1 receptor but not to other tyrosine kinases (insulin receptor, IGFR-1R). The criteria for a positive clone is a >2 fold difference vs. an unrelated target. The results of the following database searches identified mannose-binding protein associated serine protease 2 (MASP-2) as a nature partner.

Sequences of Peptide Binders to Tie-1

| | |
|---|---|
| Consensus: | GxAVVFLDRWGNP |
| >RPT13 | SLWGCSGRAVLFLDSVGNPTGTVRC |
| >RPT9 | RRVDAGGAVVYLDRWGNVSV |
| >RPT34 | VVFLDRWGNPQYLGVKASGG |
| TI1-G11-R40 | GPFSWLFETEWGNPKTVPFGADRWNRHGRWDPGPVSDYGT |

Results of Advanced Blast Search
Reference:
Altschul, Stephen F., Thomas L. Madden, Alejandro A.
Schäffer,
Jinghui Zhang, Zheng Zhang, Webb Miller, and David J.
Lipman (1997),
″Gapped BLAST and PSI-BLAST: a new generation of protein
database search
programs″, Nucleic Acids Res. 25: 3389-3402.

| | |
|---|---|
| RID: | 988980952-24595-10839 |
| Query = RPT9: | RRVDAGGAVVYLDRWGNVSV |
| | (20 letters) |
| Database: | Non-redundant SwissProt sequences |
| | 96,103 sequences; 35,068,824 total letters |

Score E
Sequences producing significant alignments:
(bits) Value
gi|7387859|sp|O00187|MASP2_HUMAN MANNAN-BINDING LECTIN
SERIN . . . 29 0.17

Alignments
>gi|7387859|sp|O00187|MAS2_HUMAN MANNAN-BINDING LECTIN
SERINE PROTEASE 2 PRECURSOR (MANNOSE-BINDING
PROTEIN ASSOCIATED SERINE PROTEASE 2) (MASP-2)
Length = 686
Score = 29.1 bits (61), Expect = 0.17
Identities = 13/25 (52%) , Positives = 16/25 (64%) , Gaps =
7/25 (28%)

| | | | |
|---|---|---|---|
| Query: | 2 | RVDAGGAVVYLD----RW---GNVS | 19 |
| | | R D+GGA+V+LD RW G VS | |
| Sbjct: | 630 | RGDSGGALVFLDSETERWFVGGIVS | 654 |

### EXAMPLE 6

### Generation of Agonist/Antagonist Assays for Tie-1 Recoptor for Determining Phenotypic Effects of Surrogates

Surrogates are tested for agonist and/or antagonist activity in cell lines expressing both full length Tie-1 and a chimeric receptor containing the extracellular domain of Tie-1 and the cytoplasmic region of the epidermal growth factor receptor (EGFR). The EGFR was chosen because: a) both the EGFR and Tie-i are receptor tyrosine kinases; b) both appear to signal following dimerization and c) there is an extensive body of information regarding EGFR signal transduction pathways and the downstream events involved in transcription and cell growth.

Several models are used including cell proliferation and gene reporter assays. In the proliferation models, full length and chimeric Tie-1 are transfected into the IL-3 dependent cell line, FDC. After selection, these cells proliferate in the presence of a putative Tie-1 agonist. In gene reporter assays, various gene-reporter systems are used, including STAT (signal transducer and activator of transcription) -luciferase, STAT-GFP (green fluorescence protein), SRE (serum response element)-luciferase and SRE-GFP. Co-transfection experiments establish cell lines expressing either full length or chimeric Tie-1. These STAT and SRE lines allow the high throughput screening of phage clones to determine the putative bioactivity of the peptide surrogates. See Carcamo, et al. Proc. Natl Acad Sci USA 95: 11146-11151.

The complete ORF of the Tie-1 gene is cloned from fetal human brain (Clontech Quick-Clone cDNA) or fetal human heart using the following primers:

| | |
|---|---|
| 5′ Tie-1 forward: | GGT CGG CCT CTG GAG TAT GOT CTG |
| 3′ Tie-1 reverse: | TCC TTG AGG CAG CTT AAG TCA GAG |

The complete ORF of the EGFR gene is cloned from the above libraries or from a placental cDNA library (Clontech Placenta Marathon ready cDNA) using the following primers:

| | |
|---|---|
| 5′ EGFR forward: | GGA GCA GCG ATG CGA CCC TC |
| 3′ EGFR reverse: | GGT CCT GGG TAT CGA AAG AGT CTG G |

In the chimeric receptor, the extracellular and transmembrane regions of Tie-1 are joined to the cytoplasmic kinase domain of the EGFR with an NHE I site which will add the amino acids alanine and serine at the junction. The primers for generating the chimeric receptor are the following primers (with the NHE site underlined):

| | |
|---|---|
| EGFR forward: | GCG CTG CTA GCC GAA GGC GCC ACA TCG TTC |
| Tie-1 reverse: | CCT GCT GCT AGC GAT GCA CAC CAG GGT TAA |
| AAGG | |

Both the full length Tie-1 and the chimeric receptor are cloned into pCDNA 3.1 for transfection experiments.

The various target cell lines are used to screen surrogate peptides with agonist and antagonist activity. The surrogates are used as peptidomimetics or for the generation of Site Directed Assays and small molecule discovery via high throughput screening.

We claim:

1. A method of identifying a naturally occurring binding partner, or binding partner precursor, for a target, said method comprising:
a) identifying an amino acid sequence motif which confers detectable binding properties of a peptide comprising said motif to a target by screening a library comprising a plurality of different expressed amino acid sequences for binding of members of said library to the target; separating members of the library which bind to the target; determining the amino acid sequence of the members of the library which bind to said target; and identifying as motifs common amino acid sequences among said determined amino acid sequences;
b) comparing the identified amino acid sequence motifs to known amino acid sequences of a genome and identifying a gene product of said genome possessing said motif as the naturally occurring binding partner, or partner precursor, for said target.

2. The method according to claim 1 wherein the target is an untranslated region of mRNA.

3. The method according to claim 1 wherein the target is a cellular receptor.

4. The method according to claim 1 wherein said library comprises a peptide library of random amino acid sequences.

5. The method according to claim 4 wherein the peptides of said library comprises a random sequence of about 10 to about 50 amino acids.

6. The method according to claim 5 wherein the random sequence comprises about 20 to 40 amino acids.

7. The method according to claim 6 wherein the random sequence consists essentially of about 20 amino acids.

8. The method according to claim 6 wherein the random sequence consists essentially of about 40 amino acids.

9. The method according to claim 1 wherein the genome is mammalian.

10. The method according to claim 9 wherein the genome is human.

11. The method according to claim 1 wherein the target is selected from the group consisting of receptors, transport proteins, transcription regulatory sites and translation regulatory sites.

12. The method according to claim 1 wherein the target comprises a protein.

13. The method according to claim 1 wherein the target comprises a nucleic acid.

14. The method according to claim 1 wherein the target is a polysaccharide.

15. The method according to claim 1 wherein the motif comprises 5 to 8 amino acids.

16. The method according to claim 15 wherein the common amino acids of said motif are contiguous.

17. A method of identifying a motif comprising an amino acid sequence of a post translational gene product wherein said motif confers detectable binding properties at a natural target of said post translational gene product, said method comprising screening a library comprising a plurality of different expressed amino acid sequences for binding of members of said library to the target; separating members of the library which bind to the target; determining the amino acid sequence of the members of the library which bind to the target; and identifying as motifs common amino acid sequences among the determined amino acid sequences of said target binding members of the library.

18. The method according to claim 17 wherein said library comprises a peptide library of random amino acid sequences.

19. The method according to claim 18 wherein the peptides of said library comprises a random sequence of about 10 to about 50 amino acids.

20. The method according to claim 19 wherein the random sequence comprises about 20 to 40 amino acids.

21. The method according to claim 20 wherein the random sequence consists essentially of about 20 amino acids.

22. The method according to claim 20 wherein the random sequence consists essentially of about 40 amino acids.

23. The method according to claim 17 wherein said library is a library derived from a primary library by fixing the identity of certain amino acids in known positions of said members of said library.

24. The method according to claim 17 wherein the common amino acids of said motif are contiguous.

25. A method for determining the activity of a gene product, said method comprising:
a) expressing said gene product in a cell;
b) contacting said cells with a ligand which binds said gene product; and
c) detecting a change in phenotype of cells in which said gene product is expressed.

26. The method according to claim 25 wherein said gene product is expressed in a plurality of different cell types.

27. The method according to claim 25 wherein said ligand possess a consensus amino acid sequence determined from a plurality of members of a peptide library which bind said gene product.

28. The method according to claim 27 wherein said ligand possesses an amino acid sequence enabling the ligand to enter said cell.

29. The method according to claim 25 wherein the change in phenotype is detected based on a change in cell growth.

30. The method according to claim 25 wherein the change in phenotype is detected based on a change in cell morphology.

31. The method according to claim 25 wherein said ligand is homologous to a natural peptide.

32. A method of determining the phenotypic outcome of the expression of a gene product comprising;
a) expressing the gene product in cells;
b) contacting said cells with an amino acid sequence comprising a motif which binds said gene product and wherein said motif is identified from members of a peptide library which bind to the target; and
c) detecting a change in phenotype of cells in which said gene product is expressed.

33. The method according to claim 32 wherein said gene product is expressed in a plurality of different cell types.

34. The method according to claim 32 wherein said amino acid sequence possesses an amino acid sequence enabling it to enter said cell.

35. The method according to claim 32 wherein the change in phenotype is detected based on a change in cell growth.

36. The method according to claim 32 wherein the change in phenotype is detected based on a change in cell morphology.

37. The method according to claim 32 wherein said motif is present in a naturally occurring gene product.

38. A method of identifying a naturally occurring binding partner, or binding partner precursor, for a target, said method comprising:
a) identifying an amino acid sequence which binds to said target by screening a library comprising a plurality of different expressed amino acid sequences for binding of members of said library to the target; separating at least one member of the library which bind to the target; determining the amino acid sequence of said member of the library which bind to said target; and
b) comparing the identified amino acid sequence of said member to known amino acid sequences of a genome and identifying a gene product of said genome possessing an amino acid sequence substantially similar to said identified amino acid sequence as the naturally occurring binding partner, or partner precursor, for said target.

39. The method according to claim 38 wherein the substantially similar amino acids are identical and contiguous.

40. The method according to claim 39 wherein at least 5 amino acids are identical and contiguous.

41. The method according to claim 38 wherein the target is an untranslated region of mRNA.

42. The method according to claim 38 wherein the target is a cellular receptor.

43. The method according to claim 38 wherein said library comprises a peptide library of random amino acid sequences.

44. The method according to claim 43 wherein the peptides of said library comprises a random sequence of about 10 to about 50 amino acids.

45. The method according to claim 44 wherein the random sequence comprises about 20 to 40 amino acids.

46. The method according to claim 45 wherein the random sequence consists essentially of about 20 amino acids.

47. The method according to claim 45 wherein the random sequence consists essentially of about 40 amino acids.

48. The method according to claim 38 wherein the genome is mammalian.

49. The method according to claim 48 wherein the genome is human.

50. The method according to claim 38 wherein the target is selected from the group consisting of receptors, transport proteins, transcription regulatory sites and translation regulatory sites.

51. The method according to claim 38 wherein the target comprises a protein.

52. The method according to claim 38 wherein the target comprises a nucleic acid.

53. The method according to claim 38 wherein the target is a polysaccharide.

54. The method according to claim 38 wherein the motif comprises 5 to 8 amino acids.

55. A method for identifying a nucleic acid sequence encoding a naturally occurring binding partner, or binding partner precursor, for a target, said method comprising:
a) identifying an amino acid sequence motif which confers detectable binding properties of a peptide comprising said motif to a target by screening a library comprising a plurality of different expressed amino acid sequences for binding of members of said library to the target; separating members of the library which bind to the target; determining the amino acid sequence of the members of the library which bind to said target; and identifying as motifs common amino acid sequences among said determined amino acid sequences;
b) comparing the identified amino acid sequence motifs to known amino acid sequences of a genome and identifying a gene product of said genome possessing said motif as the naturally occurring binding partner, or partner precursor, for said target; and
c) identifying said nucleic acid sequence encoding said naturally occurring binding partner, or partner precursor.

56. A method of identifying a nucleic acid sequence encoding a naturally occurring binding partner, or binding partner precursor, for a target, said method comprising:
a) identifying an amino acid sequence which binds to said target by screening a library comprising a plurality of different expressed amino acid sequences for binding of members of said library to the target; separating at least one member of the library which bind to the target; determining the amino acid sequence of said member of the library which bind to said target;
b) comparing the identified amino acid sequence of said member to known amino acid sequences of a genome and identifying a gene product of said genome possessing an amino acid sequence substantially similar to said identified amino acid sequence as the naturally occurring binding partner, or partner precursor, for said target; and
c) identifying said nucleic acid sequence encoding said naturally occurring binding partner, or partner precursor.
